(19) United States
(12) Patent Application Publication
(10) Pub. No.: US 2012/0259249 A1
(43) Pub. Date: Oct. 11, 2012

(54) Noninvasive Ultrasound-Based Retinal Stimulator: Ultrasonic Eye

(76) Inventors: Butrus T. Khuri-Yakub, Palo Alto, CA (US); Omer Oralkan, Morrisville, NC (US); Stephen A. Baccus, Half Moon Bay, CA (US); Michael D. Menz, San Bruno, CA (US)

(21) Appl. No.: 13/441,650

(22) Filed: Apr. 6, 2012

Related U.S. Application Data
(60) Provisional application No. 61/516,832, filed on Apr. 8, 2011.
(60) Provisional application No. 61/620,947, filed on Apr. 5, 2012.

(51) Int. Cl. A61F 9/08; A61N 7/00

(52) U.S. Cl. 601/2

(57) ABSTRACT

Focused ultrasound is a promising technology for neural stimulation that is non-invasive, and capable of passing through the skull. Here we use the isolated retina to characterize the effect of ultrasound on an intact neural circuit and compared these effects to those of visual stimulation of the same retinal ganglion cells. Ultrasound stimuli evoked precise, stable responses that looked qualitatively similar to strong visual responses but with shorter latency. We found that ultrasonic stimulation activates cells presynaptic to ganglion cells, which may include photoreceptors and interneurons. Ultrasonic stimulation is an effective and spatial-temporally precise method to activate the retina. Ultrasonic stimulation may have diagnostic potential to probe remaining retinal function in cases of photoreceptor degeneration, and therapeutic potential for use in a retinal prosthesis. In addition, ultrasound promises to be a useful tool to understand the dynamic activity in the interneuron population of the retina.

### CROSS REFERENCE TO RELATED APPLICATIONS

This application claims the benefit of U.S. provisional patent application 61/516,832, filed on Apr. 8, 2011, entitled “Noninvasive ultrasound-based retinal stimulator: ultrasonic eye”, and hereby incorporated by reference in its entirety. This application also claims the benefit of U.S. provisional patent application 61/620,947, filed on Apr. 5, 2012, entitled “Precise neural stimulation in the retina using focused ultrasound”, hereby incorporated by reference in its entirety, and hereinafter referred to as “Reference material”.

### FIELD OF THE INVENTION

This invention relates to ultrasonic stimulation of neural cells.

### SUMMARY

Focused ultrasound is a promising technology for neural stimulation that is non-invasive, and capable of passing through the skull. Here we use the isolated retina to characterize the effect of ultrasound on an intact neural circuit and compared these effects to those of visual stimulation of the same retinal ganglion cells. Ultrasound stimuli at a frequency of 43 MHz and a focal spot diameter of 90 μm were delivered from a piezoelectric transducer to the intact, isolated salamander retina. Ultrasound stimuli evoked precise, stable responses that looked qualitatively similar to strong visual responses but with shorter latency. By moving the ultrasound focus laterally we measured the receptive fields of neurons, revealing Off centers and On surrounds, evidence of retinal processing. Using white noise modulation of ultrasonic power, we found that retinal ganglion cells responded more to the transition from On to Off compared to visual stimuli. By presenting ultrasound and visual stimulation together, we found that ultrasonic stimulation altered the sensitivity to visual stimuli, but not visual temporal filtering. By blocking synaptic transmission, we found that ultrasound did not directly activate retinal ganglion cells, even at much lower or higher power levels than normally used. Ultrasound responses differed from visual responses in that many neurons responded transiently to the onset and offset of ultrasonic stimuli but responded only to the offset of visual stimuli. We conclude that ultrasonic stimulation activates cells presynaptic to ganglion cells, which may include photoreceptors and interneurons. Ultrasonic stimulation is an effective and spatial-temporally precise method to activate the retina. Because the retina is the most accessible part of the central nervous system in vivo, ultrasonic stimulation may have diagnostic potential to probe remaining retinal function in cases of photoreceptor degeneration, and therapeutic potential for use in a retinal prosthesis. In addition, ultrasound promises to be a useful tool to understand the dynamic activity in the interneuron population of the retina.

### BRIEF DESCRIPTION OF THE DRAWINGS

FIG. 1 shows an exemplary embodiment of the invention. Here a source of focused ultrasound 106 provides ultrasound 108 to a retina including a photoreceptor layer 102 and a bipolar cell/ganglion cell layer 104.

### DETAILED DESCRIPTION

### Introduction

Non-invasive neuro-stimulation with near-cellular spatial resolution has many applications for both basic research and in the clinic for diagnosis or therapy. Currently, electrical stimulation is either invasive or when used non-invasively has poor spatial resolution. Magnetic stimulation has poor spatial resolution, and optical stimulation is invasive. Focused ultrasound is a recently explored non-invasive method for neurostimulation. It can achieve high spatial resolution either by using high frequencies or by having multiple transducers focused on the same spot (Clement et al., 2005), or some combination of these. For brain stimulation, in which ultrasound must go through bone, lower frequencies (<1 MHz) are preferred. However, because the retina is an exposed part of the central nervous system, the potential exists to use higher frequencies. Currently, the mechanism of ultrasonic stimulation of neural activity is unknown, although it may be mechanical, through radiation pressure or cavitation, or possibly may involve thermal energy delivery. Similarly, the biological transduction of ultrasonic stimulation is also unknown, although it may involve mechanosensitive ion channels or synaptic release machinery (Tyler, 2011; Krasovitski et al., 2011).

For any method of perturbing neural activity, it is critical to know whether the method of stimulation activates or suppresses activity, and what are the spatial and temporal parameters of this effect. A second, often overlooked property of neural stimulation is to assess how the artificial stimulus changes the ongoing sensitivity to the circuit's natural input. For example, a stimulation method that briefly causes activity, but causes a prolonged drop in sensitivity to the circuit's input may lead to a mistaken conclusion about neural function if both effects are not understood.

Here we characterize the effects of low power, high-frequency focused ultrasound on an intact neural circuit, the salamander retina. With the isolated retina, we used a multielectrode array to compare the responses of retinal ganglion cells to ultrasound, and to light, the natural stimulus. Furthermore, we characterized how ultrasonic stimuli changed visual sensitivity. We find that ultrasonic stimulation can convey information to the retina with a temporal precision equal or greater than for visual input. We further find that ultrasonic stimulation causes brief, localized changes in spatio-temporal visual sensitivity. The measured spatial localization of the stimulation in our study was 110 μm (standard deviation), which includes the lateral spread of the retinal circuitry. We conclude that ultrasonic stimulation has potential as a tool to perturb ongoing neural activity in the retina for the study of circuit function, and may have potential for use in a noninvasive retinal prosthesis.

### Methods

### Electrophysiology

Multielectrode recordings were performed as previously described (Baccus and Meister, 2002). The isolated retina of a tiger salamander was adhered by surface tension to a dialysis membrane (˜100 μm thick) attached to a plastic holder. It was then placed on a motorized manipulator and lowered onto a 60-electrode array (ThinMEA, Multichannel Systems) ganglion cell side down. We used the low-density array (8×8 grid, 100 μm spacing) with uniform field visual stimuli and a high density array (two 5×6 grids with 30 μm electrode spacing, the grids separated by 500 μm) with the 100 μm spot visual stimuli centered over one grid.

### Ultrasound Stimulus Characteristics

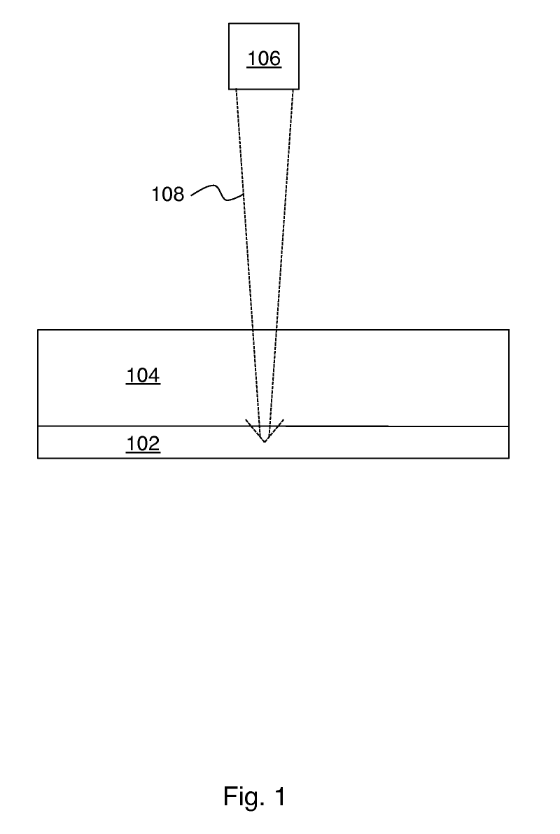

The ultrasound (US) transducer was a custom made, piezoelectric element with quartz focusing lens and 43 MHz center frequency. It was mounted on a micromanipulator (model MPC-385-2, Sutter Instruments, Novato, Calif.) and immersed in the perfusion fluid above the retina (Reference material FIG. 1b). Ultrasound waves propagated from the transducer, through the water bath, the dialysis membrane used to hold the retina down, the retina, and would reflect off the multielectrode array. Some energy was also reflected off the dialysis membrane and retina interfaces, and this could be used in ultrasound imaging mode to determine the proper depth of the transducer for retinal stimulation. A function generator (Model 8116A, Hewlett-Packard Co., Palo Alto, Calif.) was used to produce the 43 MHz carrier, which was gated on and off by the analog output from a National Instruments data acquisition board and then passed through a 50 dB RF power amplifier (Model 320L, Electronic Navigation Industries Inc., Rochester, N.Y.) to stimulate the custom transducer. The gating signal is saved on the electrophysiology recording computer so responses can be precisely timed to the stimulus. The working distance of the transducer is about 4.3 mm, with a lateral resolution estimated to be about 100 μm, and the focal length spans the retina in depth (see Reference material FIG. 1a for a simulation of the spatial power distribution). It is difficult to measure the power output of a 43 MHz transducer because the off-the-shelf hydrophones are not calibrated for that frequency and do not have sufficient spatial resolution. Therefore we measured the insertion loss from 20 MHz to 50 MHz. Power was measured at 20 MHz using a laser interferometer (model OFV-511, Polytec GmbH, Waldbronn, Germany) and we calculated the expected power density at 43 MHz using the insertion loss curve (Reference material Supplemental FIG. 1). The calculated time-averaged acoustic power is ˜30 W/cm² for 50% duty cycle stimulus (e.g., one second On, one second Off).

The 43 MHz carrier was modulated at low frequencies (0.5-15 Hz) to exactly match the temporal pattern that we use for visual stimulation (Reference material FIG. 1c). For most experiments this consisted of 1 s On time followed by a 1 s Off, repeated for many cycles, for a total duration of one to five minutes. In some experiments, the On and Off times were varied randomly to make a binary noise stimulus in which the random variation in On and Off times are a multiple of 1/30 s (picked to copy visual stimuli temporal structure, i.e., two frames of a 60 Hz monitor).

### Optimizing Ultrasound Stimulus Parameters

The literature implies that using ‘pulsed’ ultrasound (carrier modulated ˜kHz) is advantageous, (Tufail et al., 2010). In our early experiments we copied this by using high frequency modulation (5-500 kHz, 50% duty cycle) of the 43 MHz carrier (Reference material Supplemental FIG. 2a) in addition to our normal low frequency modulation. In our early experiments our goal was to get reliable, reproducible, responses that look similar to visual responses. Once this was achieved, we explored the temporal parameter stimulus space to look for a more optimal stimulus, one that was equally effective, but perhaps at lower power. One might hypothesize that there is some resonant frequency or ideal pulse length that optimally stimulates cells, allowing us to achieve strong stimulation at very low power levels. These manipulations in parameter space pertain to the high frequency modulation, which generates a pulse train that can be most easily characterized by frequency and duty cycle (i.e., percent ‘on’ time), where pulse duration is (duty cycle)*(1/f). Our manipulations of these parameters (data not shown) suggested that only average power is important, neither frequency nor pulse duration matters. By keeping the duty cycle constant (e.g., 50%) while varying the frequency over a wide range (DC to 1 MHz, by a factor of 10), the average power will remain constant, and the pulse duration co-varies with frequency. This experiment with one second “On” and one second “Off” ultrasound stimulation (0.5 Hz) clearly shows that neither frequency nor pulse duration has any effect on responses when average power is held constant (Reference material Supplemental FIG. 2b). In another experiment (data not shown) frequency was varied from 50 Hz to 500 kHz by a factor of ten to make sure we did not skip over a resonant frequency. For the DC case, there is no additional modulation of the 43 MHz carrier other than 0.5 Hz, and the amplitude is reduced so the average power is the same for all conditions. Since 10 Hz modulation is within the physiologic range, it can produce a cell response at that frequency (Reference material Supplemental FIG. 2a). When we average the normalized mean across all cells, there is no effect of frequency or pulse duration. In subsequent experiments we eliminated the high frequency modulation, as it appeared to serve no useful purpose, and made the stimulus unnecessarily more complicated. Furthermore, if there is any kind of cell damage dependent on the peak power, a continuous waveform is advantageous because it has the lowest peak power for a given average power. The absence of a frequency or pulse duration effect tells us something about the mechanism. There must be some integrating time constant that is relatively slow, in normal physiologic range (e.g., tens of milliseconds). A faster time constant (microseconds to milliseconds) would be expected to have an optimal stimulus with matching pulse duration.

### Calibration of US Transducer Orientation and Location

First, the tilt angle of the US transducer was adjusted by using the reflected signal to verify that the transducer was orthogonal to the MEA. The same US transducer used to generate the signal can also be used to detect the reflected signal. The reflected signal was displayed on an oscilloscope (Model TDS3054B, Tektronix, Beaverton, Oreg.). In order to calibrate the position of the US transducer relative to the MEA, a small pinhole (˜200 μm) in a piece of aluminum foil was positioned over the center of the array. The position of the hole over the array was confirmed by a CCD camera image. Next, the US transducer was moved in three dimensions so that the focus as measured by the reflected signal was centered over the hole. For the low-density array the calibrated transducer position is in the center of the array. For the high-density array, the transducer is positioned in the center of one array (either left or right).

### Visual Stimuli

In our early experiments visual stimuli were uniform field flashes from a red LED. In order to generate spatial stimuli, later experiments used a DLP projector (DELL model 2300 MP) focused on the retina from below. The output of the projector was attenuated by 3ND filters and adjusted so that the photopic mean intensity was close to ˜10 mW/m². Visual stimuli had the same temporal pattern (1 s On, 1 s Off, or binary random noise) as used for US stimulation to facilitate a direct comparison. We also used a spatial checkerboard with random, binary modulation of 100 μm checks. This is used to measure space-time receptive fields. In one experiment this visual stimulus was combined with periodic US stimulation to measure how ultrasound disturbs normal visual processing.

### Receptive Fields

Spatial receptive fields and temporal filters were calculated by the standard method of reverse correlation with the spatial checkerboard visual stimulus consisting of binary squares (Chichilnisky, 2001), such that

$$F ( x , y , τ ) = {\int }_{0}^{T} s ( x , y , t - τ ) r ( t ) d t , \qquad ( 1 )$$

where F(x,y,r) is the linear response filter at position (x,y) and delay τ, s(x,y,t) is the stimulus intensity at position (x,y) and time t, normalized to zero mean, r(t) is the firing rate of a cell, and T is the duration of the recording. The filter F(x,y,τ) was computed by correlating the visual stimulus to spike times for ganglion cells. A temporal filter was computed as the spatial average of F( ). For the ultrasound and visual spot binary modulation, F(x,y,τ) becomes F(τ) and s(x,y,t) becomes s(t) as there is no spatial dimension.

### Results

### Comparing Visual Responses to Ultrasound Responses (Simple Periodic Stimuli)

The responses of individual ganglion cells to a 0.5 Hz ultrasound stimulus were strong and reproducible, much like visual responses to a 0.5 Hz flashing 100 μm spot that illuminated the same area as the ultrasound stimulus (Reference material FIG. 2a). For some cells, the firing rate and duration of the responses were similar, except that latency of the ultrasound response was shorter than the visual latencies (Reference material FIG. 2a, middle and right). For other cells, ultrasound stimuli generated both ON and OFF responses, whereas visual stimuli generated only OFF responses (Reference material FIG. 2a left).

We found that ultrasound stimulation produced precisely timed spikes across multiple repetitions (Reference material FIG. 2b). Transient bursts of action potentials occurred both at the onset and offset of the US pulse. The temporal precision of this activity in some cases was smaller than 1 ms, as has been reported for visual stimuli (Berry et al., 1997).

For all cells that respond to both visual and ultrasound stimulation, the latency of the ultrasound response was equal to or shorter than the visual response (Reference material FIG. 2c Left, mean visual On latency=141 ms, ultrasound=92ms, two-tailed paired t-test p=3.1×10⁻⁵, n=14, mean visual Off=112 ms, ultrasound Off=53 ms, two-tailed paired t-test p=9.6×10⁻⁸, n=19). The On visual responses are typically longer in latency than Off responses because they are mediated by On bipolars that have a slower metabotropic glutamate receptor mechanism. Likewise, for ultrasound responses, the On latency is longer than Off latency (Reference material FIG. 2c, Right, mean visual On-Off latency=33.4 ms, same cells mean ultrasound On-Off=40.7 ms, paired two-tailed t-test p=0.38, n=11, figure shows additional ultrasound On-Off cells that do not have visual On-Off response.). The longer latency of the ultrasound On response is approximately the same as the longer latency of the visual On response, which implies a common mechanism.

We can summarize how well cells respond to the stimulus by analysis in the frequency domain. The stimulus has a fundamental frequency of 0.5 Hz. Cells that only respond to onset or offset will have a strong fundamental (F1). The second harmonic (F2) is 1 Hz, cells that respond equally strongly to both onset and offset will have a strong second harmonic but weak fundamental response. The ratio of the second harmonic to the fundamental is generally much greater for ultrasound stimuli, especially when the ratio for the visual stimuli is relatively lower (Reference material FIG. 2d, to the left of the vertical line). For cells in which the F2/F1 ratio is higher for visual stimuli (Reference material FIG. 2d, to the right of the vertical line) there does not appear to be any systematic difference between ultrasound and visual stimuli F2/F1 ratios.

These results are consistent with the hypothesis that for some cells, responses to visual and ultrasound stimulation are very similar (Reference material FIG. 2a middle-right cells); but for other cells, ultrasound is producing a response that visual stimuli are not (Reference material FIG. 2a, far left cell). These are cells that will produce a strong fundamental response to visual stimuli, but a weak fundamental for ultrasound stimuli. These results suggest that both On and Off channels are being stimulated, but the ultrasound stimulation is less specific than the visual. Therefore, ultrasound is likely stimulating some retinal circuitry that either precedes bipolar cell receptors in the outer plexiform layer (i.e., photoreceptors and/or horizontal cells) and/or the ultrasound is directly stimulating bipolar cell receptors. This result does not preclude additional stimulation downstream.

### Effect of Ultrasound Power

We wish to pick a power level that generates a robust, reproducible response similar to a visual response, but one that uses no more power than necessary. We varied average power across a wide range starting at a level that was below threshold for obtaining a response, using our standard one second “On”, one second “Off” stimulus, which is commonly used as a simple visual stimulus to classify cell types. There is considerable variability between cells when measuring peak firing rate as a function of power (Reference material FIG. 3a). Generally, they increase firing rate with power until saturation is achieved, where response may stabilize or peak firing rate may decrease. We wish to choose a value for experiments that is above threshold but below saturation. To summarize across the population, peak firing rates are normalized to the maximum for each cell (Reference material FIG. 3b). At 30 W/cm², the responses appear to be at a maximum, and higher power does not generate higher firing rates on average. We choose 30 W/cm² as our default power level for most experiments. At this power level we measured heating with a thermocouple at about 0.5° C. The latency varies strongly with power for some cells. Low power can produce very long latencies while short ultrasound latencies are associated with higher power (see Reference material FIG. 3c for a single cell example).

### Ultrasound Mapping of Receptive Fields

The responses to ultrasound stimulation are generally remarkably similar to visual responses, with apparent activation of On and Off channels. A visual spot stimulus can be moved to different spatial locations to map the spatial receptive field of a cell, which typical has a center/surround antagonism. Most commonly, the center responds to offset of the stimulus, (Off), and the surround to onset, (On). The ultrasound transducer can be moved laterally to map the receptive fields of ganglion cells (Reference material FIG. 4). The transducer was moved in relatively large steps (350 μm) because the surround is typically quite large. The center of the receptive field can be several hundred microns from the center of the array. In the two examples shown, the ultrasound stimulus demonstrates a clear center/surround organization (Reference material FIG. 4a,b). The center has a strong Off response and weak On response, while the surround region responds to onset of the stimulus, but not offset. As is the case with visual stimuli, the On-surround is a larger region compared to the Off-center (see Reference material FIG. 4c for a population summary. On width is always bigger or equal to Off width). The spatial resolution of the ultrasound transducer is something better than 350 μm (see Perturbing normal visual processing with an ultrasound stimulus and Reference material FIG. 6d for a better measurement of spatial resolution).

Modeling the Response with a Linear Filter and Static Nonlinearity

The function of retinal ganglion cells can be modeled as a linear filter followed by a static non-linearity. Phenomenon such as adaptation or a gain change can be viewed as changes in the model parameters. A Gaussian white noise stimulus is generally used to calculate the model, but binary noise can be used instead. The ultrasound stimulus was modulated in time with binary noise and a linear/non-linear model was calculated. This can be compared to modulating a 100 μm spot visual stimulus with the same binary noise and calculating the model with the visual stimulus. The linear filter is calculated from the spike-triggered average, which is the average stimulus that precedes a spike. After convolving the stimulus through this filter, a static nonlinearity describes the relationship between the filter output and firing rate (Reference material FIG. 5a). The filters are normalized so that all the gain or sensitivity is expressed in the slope of the non-linearity along with a threshold, while the filter describes the kinetics, (i.e., On vs. Off, latency, differentiating vs. integrating).

The ultrasound filters (in red, Reference material FIG. 5a, left) have very short latencies compared to the visual filters, which is expected. The filters are also very strongly biphasic, even triphasic, which means they are differentiating or high-pass filters, responding to the transition from On to Off. Sometimes the ultrasound filter will have the opposite polarity as the visual filter (Reference material FIG. 5a, middle). This kernel reversal is consistent with different strengths of On and Off responses from ultrasound compared to visual stimuli (Reference material FIG. 2a, left). For ganglion cells that receive inputs from both On and Off bipolar cells, different stimuli could excite these pathways with different relative strengths, and this can occur naturally with visual stimulation alone (Geffen et al., 2007). It could also be explained in terms of antagonistic center/surround receptive field structure. One stimulus might preferentially stimulate the center, while the other stimulates the surround more. For one On cell (Reference material FIG. 5a, bottom), the ultrasound and visual filters have a more similar shape, and the difference is primarily in the latency. There is more diversity in the nonlinearities (Reference material FIG. 5a, right). In general, the gain for ultrasound response can be greater (Reference material FIG. 5a, top), less than (Reference material FIG. 5a, bottom), or about equal to the gain for visual response. There can also be shifts in the threshold (Reference material FIG. 5. a, bottom). These shifts in threshold and gain are within the physiologic range of shifts that occur with contrast adaptation (Baccus and Meister, 2002).

One hypothesis is that ultrasound is stimulating photoreceptors only, and the only difference with visual stimulation comes from bypassing the phototransduction cascade, yielding shorter latencies than visual stimuli. If this were true, then the differences in the visual and ultrasound filters could be explained by another fixed linear filter (i.e., does not vary from cell to cell). In this case, we would expect the visual and ultrasound filter parameters to be correlated. For that purpose, we summarize filter characteristics across the population in Reference material FIGS. 5b, c and d, looking at latency to first peak (Reference material FIG. 5b), peak frequency measured from the FFT of the filter (Reference material FIG. 5c), and the weights of the first two principal components (Reference material FIG. 5d). For Off cells, visual latencies are more diverse than ultrasound latencies, there is not a single number to describe the difference, although there is a weak positive correlation (Reference material FIG. 5b, Pearsons correlation coefficient r=0.43). The peak frequencies of the ultrasound filters are consistently higher than visual stimulation. For Off cells the range of ultrasound peak frequencies is broad while the visual filters have a narrow range (Reference material FIG. 5c, r=0.53). Principal components analysis was performed separately on the Off visual and ultrasound filters, and about 95% of the variance is explained by the first two principal components in both cases. The differences in the weights (ultrasound-visual) of the first principal components are relatively small while the differences in the second principal component weights are much larger and uncorrelated (Reference material FIG. 5d, r=0.13). Finally, the single On cell has a filter shape that is similar for both visual and ultrasound stimuli, but with shorter latency and higher frequency (Reference material FIG. 5b,c). Considering all the results shown in Reference material FIG. 5, the variance between cells is too great for any one filter (i.e., phototransduction cascade) to account for the differences in the ultrasound and visual filters. Therefore, stimulation of only photoreceptors is unlikely.

Perturbing Normal Visual Processing with an Ultrasound Stimulus

The results presented so far have compared single cell responses to ultrasound and visual stimuli. By applying both visual and ultrasound stimuli simultaneously we can determine how ultrasound perturbs the normal processing of a visual signal. For this experiment the visual stimulus is a binary random checkerboard, from which we can extract the linear temporal filter, the static nonlinearity, and the two dimensional spatial receptive field (with the low-density array (˜1×1 mm), so receptive fields cover a large area,). During this visual stimulation the ultrasound delivers a periodic pulse of 200 ms duration every 2 seconds (Reference material FIG. 6a). The data is analyzed by correlating response to the visual stimulus, but it is subdivided into three time epochs, part one is the 200 ms during which the ultrasound pulse is turned on (‘On’), part two is the 200 ms immediately after the ultrasound pulse is turned off (‘Off’), and part three is a control period that extends from 300 ms after the pulse is turned off until the next pulse (‘Control’). These Off and Control periods were defined by first analyzing in multiple 200 ms epochs, and it was found that these three time epochs were sufficient to capture the dynamic changes. For cells that have their receptive fields close to the ultrasound transducer, the effect of ultrasound is fairly consistent. During ultrasound stimulation cells exhibit either reduced firing, or no change from control, but immediately after ultrasound is turned off, there is a brief increase in firing rate (Reference material FIG. 6b). There is virtually no change in the temporal filters (some will be noisier because of reduced firing rate). There are changes in the nonlinearities consistent with the changes in firing rate. The cells near the ultrasound focus both have lowered thresholds and higher gains during the Off period compared to the On period. For cells that are further from the transducer, there are a greater variety of effects on firing rate and the nonlinearities. This greater diversity of effects may simply reflect the fact that there are very few cells close to the transducer and many more cells further away from the transducer. Another hypothesis is that when the US transducer focus is close to the cell RF, the effect of ultrasound is in the center of the RF; and when the US focus is farther away, the ultrasound could be modulating the center or surround of the RF. For the group of cells that are further away from the US focus, we present two examples of different effects. One cell (Top) demonstrates a decrease in firing rate during the Off period. The threshold of the Off period nonlinearity is increased, although the gain appears unchanged. Another cell (Bottom) exhibits a complete suppression of firing during ultrasound stimulation. Nonetheless, the temporal filters are again unchanged and nonlinearities are consistent with changes in firing rate. In summary, the ultrasound perturbation generally does not fundamentally change the signal processing (exception is the one cell (out of 35) where spiking is completely suppressed by ultrasound), but appears to shift thresholds and gain in a manner similar to normal gain control mechanisms in the retina (Baccus and Meister, 2002).

We can use the spatial receptive field maps to derive an upper limit on the spatial resolution of the ultrasound transducer. A 2d Gaussian fit to the spatial receptive fields shows the spatial distribution of RFs relative to the ultrasound transducer location (black ‘+’, Reference material FIG. 6c). If we take the RMS of each RF and sum across all RFs, we get the total sensitivity map (Reference material FIG. 6c). For each cell, we calculate the slope of the nonlinearity as an estimate of the sensitivity for the three conditions (On, Off, Control). The RMS of each receptive field is weighted by the change in sensitivity created by ultrasound On or Off conditions compared to Control, and these results are summed across all cells. This yields a spatial map of ultrasound sensitivity effects. For the ‘On’ effects, there are two groups of cells affected, those nearby the transducer show a reduction in sensitivity, but there are cells far away that experience the opposite effect, an increase in sensitivity. For these distant cells, ultrasound is likely stimulating the surrounds of these cells. The Off effect, in increase in sensitivity, appears more localized (Reference material FIG. 6c). These 2d maps can be converted into 1d graphs of change in sensitivity vs. distance from the transducer (Reference material FIG. 6d). A Gaussian fit to the Off effect produces a standard deviation of 110 μm, which can be considered an upper limit on the spatial resolution of the ultrasound transducer. This measure of resolution is affected by the lateral spread of the signal inherent in retinal circuitry, so the actual spatial resolution must be smaller.

Effects of Ultrasound when Synaptic Transmission is Blocked

With the results presented so far we have seen that responses to ultrasound stimuli are remarkably similar to visual stimuli, implying that considerable retinal processing is involved, and that we are not exclusively stimulating ganglion cells directly. In order to directly measure the effect of ultrasound on ganglion cells, we blocked vesicular transmitter release. This was accomplished by perfusing the retina with 100 μM CdCl₂ and MgCl₂ replaced CaCl₂ (Brivanlou et al., 1998). The Cd blocks calcium channels and substituting Mg for Ca maintains a high level of spontaneous activity (higher than normal). The ultrasound stimulation consists of one second On and one second Off for 60 seconds. Before perfusing CdCl₂, we measured responses to the 100 μm visual spot and ultrasound stimuli at the normal power level (30 W/cm²) as a control to verify normal stimulation (Reference material FIG. 7a).

While perfusing CdCl₂, which abolishes synaptic transmission, ultrasound stimulation (@30 W/cm²) does not produce strong, precisely timed, reproducible responses (Reference material FIG. 7b). In fact, there is no response. The stimulus was repeated at progressively higher power levels (up to 180 W/cm²), but at no point did we obtain any stimulus-locked response, at most there was some modulation of spontaneous activity. A summary of the second harmonic (the strongest stimulus-correlated response for ultrasound stimuli, see Reference material FIG. 2d) of 19 cells shows that none of these cells responded to ultrasound stimuli in the presence of CdCl₂. Thus, the direct effect of ultrasound stimulation on ganglion cells is not neural stimulation resulting in an action potential.

It's a bit surprising that ultrasound is not directly stimulating ganglion cells. If the mechanism of ultrasound stimulation involved something like direct activation of Na channels (Tyler et al., 2008), then we expect ganglion cells to be stimulated in this experiment. One possible, although unlikely explanation, is that ultrasound selectively activates Ca channels, and Cd now blocks these Ca channels. An alternative explanation is that ultrasound stimulation has its primary effect early in the visual system (photoreceptors, horizontal cells, bipolar cells) and this is amplified by retinal circuitry and synapses.

### Discussion

We have shown that ultrasound stimulation produces precise, stable responses qualitatively similar to visual responses. High-frequency, focused ultrasound can achieve fine lateral spatial resolution (˜100 μm) in retinal neurostimulation. Ultrasound stimulation during visual stimulation changes visual sensitivity. With regard to the optimal stimulus parameters, we find high-frequency modulation to be unnecessary, neither beneficial nor detrimental. The low-frequency modulation should be in the normal physiologic range equivalent to the natural stimulus. One result that is consistent across the literature is that lower average power levels are associated with excitation, higher powers with suppression, and still higher powers with cell damage. Thus, for any given preparation, the average power used should be experimentally determined by starting at levels that are below threshold and gradually increased until the desired effect is obtained.

Early experiments demonstrated the effectiveness of ultrasound suppression of neural activity, but not for generating activity. Using ultrasound, it was possible to reversibly suppress neural activity in cat visual cortex by applying ultrasound to the LGN (Fry et al., 1958) and reversibly block conduction in cat saphenous nerve(Young and Henneman, 1961). An early study in cat and rabbit brain emphasized that neural excitation occurred at relatively lower ultrasound power levels while higher power levels produced suppression (yelling and Shklyaruk, 1988). In some brain slice experiments in the 1990's, the effect of ultrasound on electrical stimulation was measured, and the predominant effect was suppression (Rinaldi et al., 1991), and the only enhancing effect could be reproduced by raising the bath temperature (Bachtold et al., 1998). In similar experiments with frog sciatic nerve, ultrasound stimulation at high power decreased compound action potential amplitudes elicited by electrical stimulation, and this was attributed to increases in temperature (Tsui et al., 2005). However, at lower power they were able to get a small enhancement (8%) in amplitude, which did not appear to be due to temperature.

However, recent studies have demonstrated the potential for ultrasound to generate neural activity. Low intensity unfocused ultrasound stimulated neurons in mouse hippocampal slices and mouse brains (Tyler et al., 2008). Responses were measured with Na and Ca imaging. In another study, low power ultrasound stimulated motor cortex in mice as measured by local field potentials and multi-unit activity in primary motor cortex (Tufail et al., 2010). They also measured EMGs and observed muscle contractions in response to ultrasound stimulation. While these studies clearly demonstrate neuro-stimulation, there was little control over the spatial extent of the stimulation, which is generally quite large (˜2 mm lateral). In a similar study using ‘focused’ ultrasound that activated rabbit motor cortex measured by fMRI and behavior, the size of the acoustic focus was estimated to be 2.3 mm in diameter and 5.5 mm in length (Yoo et al., 2011).

### Where in the Retina is the Stimulation Occurring and What Can We Say About Mechanism?

The strong similarity between ultrasound and visual responses, the presence of On and Off components with Off responses having shorter latencies than On (Reference material FIG. 2), the center/surround receptive field structure measured with ultrasound (Reference material FIG. 4), all indicate that retinal circuitry is processing the ultrasound signal. The origin of the On response being slower than the Off response comes from metabotropic glutamate receptors on the On bipolar cells in the outer plexiform layer. It is reasonable that there is at least some stimulation that either occurs at this receptor or precedes it (photoreceptors or horizontal cells). Furthermore, we know that ultrasound did not directly stimulate ganglion cells (Reference material FIG. 7) when synaptic transmission is blocked. Is it possible that all of our results can be explained if ultrasound is stimulating photoreceptors exclusively? This is an important question on several levels, if ultrasound stimulation is to be of value in clinical ophthalmology, either as a diagnostic tool or as a prosthetic, it must stimulate something in the retina beyond photoreceptors or it will have no advantage over light stimulation. Our primary result that suggests ultrasound stimulation involves more than photoreceptors is the observation that the visual and ultrasound linear filters that characterize the kinetics of stimulation are not consistent with one fixed low-pass filter (i.e., model of phototransduction cascade) preceding the ultrasound filter to make the visual filter (Reference material FIG. 5b).

Another result in which ultrasound stimulation is different from visual stimulation is when some cells exhibit a strong onset response to ultrasound whereas the visual response has no onset response (Reference material FIG. 2a, Left). This also implies that cells beyond photoreceptors are activated by the ultrasound stimulus. This response to both the onset and offset of ultrasound could arise if multiple pathways to the ganglion cell were activated. For example, if both On and Off bipolar cells were directly depolarized by ultrasound, and the On bipolar cell signal first passed through an intervening inhibitory amacrine cell synapse, then both onsets and offsets of ultrasound would generate a net excitation in the ganglion cell.

In all the previous studies that show stimulation, the preparation is either brain slice or the whole brain (yelling and Shklyaruk, 1988; Tyler et al., 2008; Tufail et al., 2010; Yoo et al., 2011). Photoreceptors are not necessary for neuro-stimulation; however, the stimulation effects of ultrasound may require an amplifying neural circuit. With optical imaging Tyler et al. (Tyler et al., 2008) noted that only 30% of the cells in the field of view were active, even though they are using unfocused ultrasound which was likely stimulating all the cells in the field. Thus, small changes in membrane potential may be amplified by synaptic transmission in the retina.

For both clinical applications and for use in the study of the retinal circuit, it will be important to determine if ultrasound can stimulate interneurons so as to cause ganglion cell spiking. Higher ultrasound frequencies and an array of focused transducers will be useful to achieve single cell stimulation. Furthermore, understanding the biophysical mechanism by which ultrasonic stimulation is converted into a change in membrane potential will be important in the design and use of devices for ultrasonic neurostimulation.

### REFERENCES

Baccus, S. A., and Meister, M. (2002). Fast and slow contrast adaptation in retinal circuitry. Neuron 36, 909-919.
Bachtold, M. R., Rinaldi, P. C., Jones, J. P., Reines, F., and Price, L. R. (1998). Focused ultrasound modifications of neural circuit activity in a mammalian brain. Ultrasound Med Biol 24, 557-565.
Berry, M. J., Warland, D. K., and Meister, M. (1997). The structure and precision of retinal spike trains. Proc. Natl. Acad. Sci. U.S.A. 94, 5411-5416.
Brivanlou, I. H., Warland, D. K., and Meister, M. (1998). Mechanisms of concerted firing among retinal ganglion cells. Neuron 20, 527-539.
Chichilnisky, E. J. (2001). A simple white noise analysis of neuronal light responses. Network 12, 199-213.
Clement, G. T., White, P. J., King, R. L., McDannold, N., and Hynynen, K. (2005). A magnetic resonance imaging-compatible, large-scale array for trans-skull ultrasound surgery and therapy. J Ultrasound Med 24, 1117-1125.
Fry, F. J., Ades, H. W., and Fry, W. J. (1958). Production of reversible changes in the central nervous system by ultrasound. Science 127, 83-84.
Geffen, M. N., de Vries, S. E. J., and Meister, M. (2007). Retinal ganglion cells can rapidly change polarity from Off to On. PLoS Biol. 5, e65.
Krasovitski, B., Frenkel, V., Shoham, S., and Kimmel, E. (2011). Intramembrane cavitation as a unifying mechanism for ultrasound-induced bioeffects. Proc. Natl. Acad. Sci. U.S.A. 108, 3258-3263.
Rinaldi, P. C., Jones, J. P., Reines, F., and Price, L. R. (1991). Modification by focused ultrasound pulses of electrically evoked responses from an in vitro hippocampal preparation. Brain Res. 558, 36-42.
Tsui, P.-H., Wang, S.-H., and Huang, C.-C. (2005). In vitro effects of ultrasound with different energies on the conduction properties of neural tissue. Ultrasonics 43, 560-565.
Tufail, Y., Matyushov, A., Baldwin, N., Tauchmann, M. L., Georges, J., Yoshihiro, A., Tillery, S. I. H., and Tyler, W. J. (2010). Transcranial pulsed ultrasound stimulates intact brain circuits. Neuron 66, 681-694.
Tyler, W. J. (2011). Noninvasive neuromodulation with ultrasound? A continuum mechanics hypothesis. Neuroscientist 17, 25-36.
Tyler, W. J., Tufail, Y., Finsterwald, M., Tauchmann, M. L., Olson, E. J., and Majestic, C. (2008). Remote excitation of neuronal circuits using low-intensity, low-frequency ultrasound. PLoS ONE 3, e3511.
Velling, V. A., and Shklyaruk, S. P. (1988). Modulation of the functional state of the brain with the aid of focused ultrasonic action. Neurosci. Behay. Physiol. 18, 369-375.
Yoo, S.-S., Bystritsky, A., Lee, J.-H., Zhang, Y., Fischer, K., Min, B.-K., McDannold, N. J., Pascual-Leone, A., and Jolesz, F. A. (2011). Focused ultrasound modulates region-specific brain activity. Neuroimage 56, 1267-1275.
Young, R. R., and Henneman, E. (1961). Functional effects of focused ultrasound on mammalian nerves. Science 134, 1521-1522.

1. Apparatus for providing stimulation to neural tissue, the apparatus comprising:
a source of focused ultrasound capable of delivering focused ultrasound to neural tissue;
wherein the source is configured to provide focused ultrasound pulses having a timing precision of 20 ms or better;
wherein the source is configured to modulate the focused ultrasound with an information bandwidth between about 1 Hz and about 5 Hz.
